(19) United States
(12) Patent Application Publication
(10) Pub. No.: US 2006/0247718 A1
(43) Pub. Date: Nov. 2, 2006

(54) Dual mode electrical stimulation to treat obesity

(75) Inventor: Warren L. Starkebaum, Plymouth, MN (US)

Correspondence Address: SHUMAKER & SIEFFERT, P. A., 8425 SEASONS PARKWAY, SUITE 105, ST. PAUL, MN, 55125, US

(73) Assignee: Medtronic, Inc., Minneapolis, MN (US)

(21) Appl. No.: 11/117,061

(22) Filed: Apr. 28, 2005

(51) Int. Cl. A61N 1/18

(52) U.S. Cl. 607/40

(57) ABSTRACT

The disclosure describes techniques for electrical stimulation to regulate the caloric intake of a patient and thereby alleviate obesity. A first set of stimulation pulses is delivered to the stomach to suppress appetite and limit food intake, and a second set of stimulation pulses is delivered to the small intestine to accelerate food transit and reduce caloric absorption. In this manner, the electrical stimulation limits food intake and caloric absorption, providing a two-pronged therapy for obesity. In a sense, the electrical stimulation electrically mimics the physiological effects of the Roux-en-Y gastric bypass procedure without the need for surgery.

### TECHNICAL FIELD

The invention relates to implantable medical devices and, more particularly, implantable gastrointestinal stimulators.

### BACKGROUND

Obesity is a serious health problem for many people. Patients who are overweight often have problems with mobility, sleep, high blood pressure, and high cholesterol. Some other serious risks also include diabetes, cardiac arrest, stroke, kidney failure, and mortality. In addition, an obese patient may experience psychological problems associated with health concerns, social anxiety, and generally poor quality of life.

Certain diseases or conditions can contribute to additional weight gain in the form of fat, or adipose tissue. However, healthy people may also become overweight as a net result of excess energy consumption and insufficient energy expenditure. Reversal of obesity is possible but difficult. Once the patient expends more energy than is consumed, the body will begin to use the energy stored in the adipose tissue. This process will slowly remove the excess fat from the patient and lead to better health. Some patients require intervention to help them overcome their obesity. In these severe cases, nutritional supplements, prescription drugs, or intense diet and exercise programs may not be effective.

Surgical intervention is a last resort treatment for some obese patients who are considered morbidly obese. One common surgical technique is the Roux-en-Y gastric bypass surgery. In this technique, the surgeon staples or sutures off a large section of the stomach to leave a small pouch that holds food. Next, the surgeon severs the small intestine at approximately mid length and attaches the distal section of the small intestine to the pouch portion of the stomach. This procedure limits the amount of food the patient can ingest to a few ounces, and limits the amount of time that ingested food may be absorbed through the shorter length of the small intestine. While this surgical technique may be very effective, it poses significant risks of unwanted side effects, malnutrition, and death.

### SUMMARY

The invention is directed to techniques for electrical stimulation to regulate the caloric intake of a patient and thereby alleviate obesity. A first set of stimulation pulses is delivered to the stomach to suppress appetite and limit food intake, and a second set of stimulation pulses is delivered to the small intestine to accelerate food transit and reduce caloric absorption. In this manner, the electrical stimulation limits food intake and caloric absorption, providing a two-pronged therapy for obesity. In a sense, the electrical stimulation electrically mimics the physiological effects of the Roux-en-Y gastric bypass procedure without the need for surgery.

Obesity is an increasing problem for many people, as individuals are consuming more calories and exercising less frequently than necessary to maintain body weight. In some cases, traditional methods for reducing body weight in obese patients may be ineffective, impractical, or potentially dangerous. Electrical stimulation of the stomach may be effective in reducing the desire of the patient to eat by inducing a feeling of fullness or nausea. In addition, electrical stimulation of the small intestine may be effective in reducing food absorption by moving the food through the small intestine more quickly.

The first and second sets of stimulation pulses may be delivered in different modes to best fit the needs of a patient. For example, the first and second sets of stimulation pulses may be applied continuously, applied in asynchronous bursts, or applied in synchronous bursts. In addition, one set of stimulation pulses may be configured to provide more vigorous stimulation than the other set of stimulation pulses. For example, some patients may respond to more vigorous stimulation of the stomach, while other patients may respond to more vigorous stimulation of the small intestine. In either case, the combined effects of the first and second sets of stimulation can contribute to an overall effect in reducing caloric intake, and thereby treating obesity.

In one embodiment, the invention provides a method for electrical stimulation of a gastrointestinal tract of a patient, the method comprising generating a first set of electrical stimulation pulses, generating a second set of electrical stimulation pulses, applying the first set of pulses to a stomach of the patient to limit food intake, and applying the second set of pulses to a small intestine of the patient to increase motility.

In another embodiment, the invention provides a device for electrical stimulation of a gastrointestinal tract of a patient, the device comprising a first lead carrying a first electrode, a second lead carrying a second electrode, and a pulse generator that delivers a first set of electrical stimulation pulses via the first lead and a second set of electrical stimulation pulses via the second lead. The first set of pulses are formulated for delivery to a stomach of the patient to limit food intake. The second set of pulses are formulated for delivery to a small intestine of the patient to increase motility.

In an additional embodiment, the invention provides a device for electrical stimulation of a gastrointestinal tract of a patient, the device comprising means for generating a first set of electrical stimulation pulses, means for generating a second set of electrical stimulation pulses, means for applying the first set of pulses to a stomach of the patient to limit food intake, and means for applying the second set of pulses to a small intestine of the patient to increase motility.

Although the stomach may be stimulated to limit the ingestion of food, the stimulation directed to the stomach also may be configured to promote faster movement into the small intestine. By reducing the amount of time the ingested food is in the gastrointestinal tract, absorbed calories may be reduced. Regulation of stimulation may be managed by the patient or clinician through the use of an external programmer which communicates wirelessly to the implantable stimulator.

In various embodiments, the invention may provide one or more advantages. For example, the delivery of electrical stimulation to the stomach may cause a sensation of fullness or nausea that prevents a patient from ingesting food. When combined with small intestine stimulation to promote motility and decreased caloric absorption, this technique for treating obesity may provide an opportunity for some patients to lose dangerous excess fat without the potential dangers associated with current surgical techniques.

The application of continuous pulses to the stomach and small intestine may allow the smooth muscle of these organs to depolarize more easily and contract with more force and at a faster rate. To accommodate certain patient conditions, the pulses may be delivered in bursts. Bursts of pulses may provide improved stimulation to depolarize the smooth muscle cells while enabling longer battery life of the pulse generator. In some patients, stimulation may provide a form of biofeedback that conditions the patient to no longer desire excess food consumption. Biofeedback conditioning of the patient could lead to reduced dependency on the stimulation, modification of the therapy, and eventual discontinuation of treatment.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a schematic diagram illustrating an implantable stimulation system for delivering electrical stimulation to the stomach and small intestine.

FIG. 2 is a schematic diagram illustrating a variety of stimulation sites on the stomach and the small intestine.

FIG. 3 is a functional block diagram illustrating various components of an exemplary implantable stimulator with multiple pulse generators.

FIG. 4 is a functional block diagram illustrating various components of an exemplary implantable stimulator with a single pulse generator.

FIG. 5 is a timing diagram illustrating continuous delivery of stimulation on two separate channels to the stomach and small intestine.

FIG. 6 is a timing diagram illustrating asynchronous delivery of stimulation on two separate channels to the stomach and small intestine, with one channel delivering bursts of pulses.

FIG. 7 is a timing diagram illustrating asynchronous delivery of stimulation on two separate channels to the stomach and small intestine, with both channels delivering bursts of pulses.

FIG. 8 is a timing diagram illustrating synchronous delivery of stimulation on two separate channels to the stomach and small intestine, with both channels delivering bursts of pulses.

### DETAILED DESCRIPTION

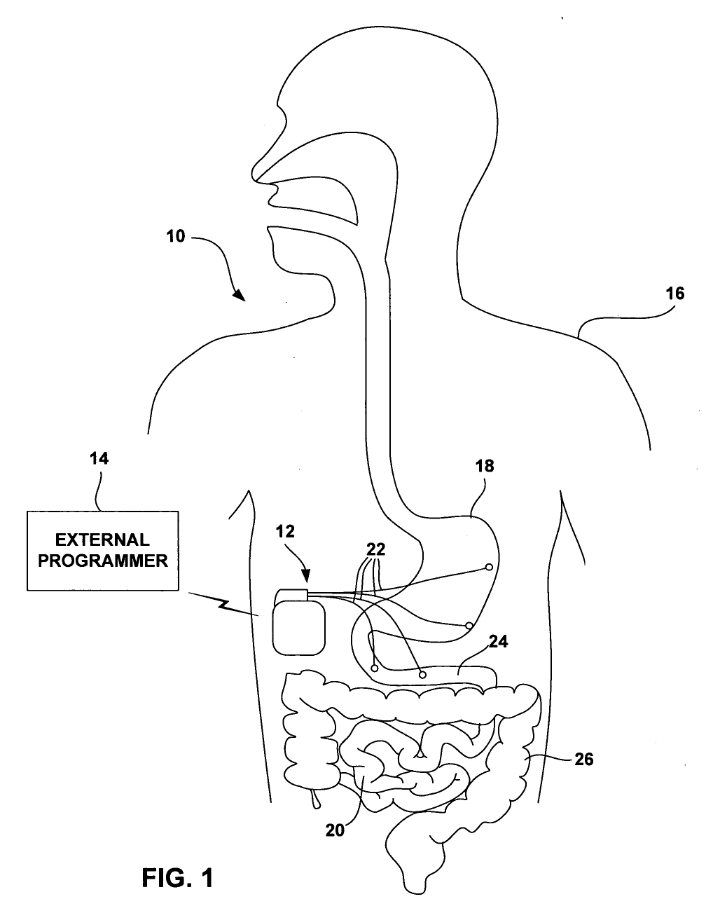

FIG. 1 is a schematic diagram illustrating an implantable stimulation system 10 for treating obesity. System 10 delivers electrical stimulation to regulate caloric intake and thereby alleviate obesity. As shown in FIG. 1, system 10 may include an implantable stimulator 12 and external programmer 14 shown in conjunction with a patient 16. Implantable stimulator 12 generates electrical stimulation pulses which are carried away from the pulse generator to stimulation sites on stomach 18 and small intestine 20 by a plurality of leads 22. In the example of FIG. 1, two leads 22 extend to stimulation sites on duodenum 24, which forms the proximal segment of small intestine 20. Other portions of small intestine 20, such as the jejunum and ileum, may be similarly stimulated. FIG. I also shows large intestine 26. Each lead 22 carries one or more electrodes that terminate in tissue at the outer lining of the stomach 14 and small intestine 20.

Implantable stimulator 12 delivers at least two different sets of stimulation pulses to stomach 18 and small intestine 20. A first set of stimulation pulses is delivered to the stomach 18 to suppress appetite and limit food intake. A second set of stimulation pulses is delivered to the small intestine 20 to accelerate food transit and reduce caloric absorption. In this manner, the electrical stimulation limits food intake and caloric absorption, providing a two-pronged therapy for obesity. In a sense, the electrical stimulation delivered by stimulator 12 electrically mimics the physiological effects of the Roux-en-Y gastric bypass procedure without the need for surgery.

Implantable stimulator 12 may be constructed with a biocompatible housing, such as titanium or stainless steel, and is surgically implanted into patient 12 at a location in which the leads 22 can access stimulation sites on stomach 18 and small intestine 20. The implantation site may be a subcutaneous location in the side of the lower abdomen or the side of the lower back. Stimulator 12 includes a pulse generator having components suitable for generation of at least two different sets of stimulation pulses. Electrical leads 22 may be surgically or percutaneously tunneled to their respective intended sites. The proximal ends of leads 22 are connected to the pulse generator to conduct the stimulation pulses to stomach 14 and duodenum 16.

In the example of FIG. 1, two leads 22 extend to stomach 18, while two other leads extend to duodenum 24. Each lead 22 may carry a single electrode or multiple electrodes. Electrode polarities may be established such that each lead 22 carries a bipolar or multipolar set of electrodes, or such that an electrode on one lead forms a bipolar arrangement with an electrode on another lead, or with an electrode formed on stimulator 12 in an “active can” arrangement. Accordingly, system 10 may include two or more leads 22, and the depiction of four leads in FIG. 1 is for purposes of example, and not limitation.

Pulse generator 12 generates a first set of stimulation pulses for stomach 18. The first set of stimulation pulses is characterized by stimulation parameters, such as amplitude, pulse width and pulse rate, selected to suppress appetite in the patient 16, e.g., by inducing a feeling of fullness or nausea. Pulse generator 12 also generates a second set of stimulation pulses for small intestine 20. The second set of stimulation pulses is characterized by stimulation parameters, such as amplitude, pulse width and pulse rate, selected to increase gastric motility through the small intestine, i.e., accelerate food transit, and thereby reduce caloric absorption in the small intestine. One or both of the first and second stimulation pulses also may be characterized by applicable burst rates and burst durations, for embodiments in which the pulses are delivered in bursts. The burst rates and burst durations may be adjusted by gating a continuous pulse output on and off at appropriate times

The first and second sets of stimulation pulses may have substantially different stimulation parameters. The differences in the pulse parameters may be a function of the diverse effects that they are intended to produce in patient 16. Again, with respect to the stomach 18, the first set of stimulation pulses may be configured to induce sensations of fullness or feelings of nausea. These induced feelings are intended to discourage the patient from consuming food, and thereby limit food intake For the amount of food that is consumed by the patient, however, the second set of stimulation pulses may be configured to increase food movement through small intestine 20. In particular, this faster movement may decrease the ability of small intestine 20 to absorb sugars and fats from the passing food. The combination of limited food intake and reduced absorption of calories promote more rapid weight loss.

Implantable stimulator 12 may include telemetry electronics to communicate with external programmer 14. External programmer 14 may be a small, battery-powered, portable device that may accompany patient 12 throughout a daily routine. Programmer 14 may have a simple user interface, such as a button or keypad, and a display or lights. In some embodiments, patient 12 may be permitted start or stop stimulation, or adjust stimulation parameters, via programmer 14.

For example, stimulation intensity associated with stimulation delivered to stomach 18 may be adjusted to increase or decrease feelings of fullness or nausea in the stomach. Similarly, stimulation intensity associated with stimulation delivered to small intestine 20 may be adjusted to increase or decrease motility in the small intestine 20. When patient 16 experiences an urge to consume food, for example, the patient may be able to elect an increased period of stimulation to help overcome the urge. Additionally, the programmer may cease stimulation if it is causing extreme discomfort or the patient is located in an environment where stimulation should be temporarily stopped. For example, the patient may turn off stimulation at night to make it easier to sleep. In each case, programmer 14 causes implantable stimulator 24 to change stimulation parameters when necessary.

External programmer 14 may be a hand-held device, as described above, or it may be embodied as a larger, more full-featured device for use by a clinician in programming implantable stimulator 12. A clinician programmer may include more features, including complete parameter modifications, firmware upgrades, data recovery, or battery recharging, if applicable.

In the example of FIG. 1, system 10 includes a single stimulator 12 with multiple leads 22 and electrodes. In other embodiments, system 10 may include multiple implantable stimulators 12 to stimulate a variety of regions of stomach 18 and small intestine 20. Stimulation delivered by the multiple stimulators may be coordinated in a synchronized manner, or performed without communication between stimulators. Also, the stimulators may be located in a variety of locations relative to the stomach 18 or small intestine 20 dependent on the particular therapy or the condition of patient 12.

The electrodes carried at the distal end of each lead 22 may be attached to the wall of stomach 18 or small intestine 20 in a variety of ways. For example, the electrodes may be surgically sutured into the outer wall of stomach 18 or small intestine 20 or fixed by penetration of anchoring devices, such as hooks, barbs or helical structures, within the tissue of stomach 18 or small intestine 20. Electrodes may also be attached to the surface of the stomach 18 or small intestine 20 using surgical adhesives, clips or sutures. In any event, each electrode is implanted in acceptable electrical contact with the smooth muscle cells within the wall of stomach 18 and small intestine 20, or on the serosal surface of the stomach or small intestine, within the muscle wall of the stomach or small intestine, or within the mucosal or submucosal region of the stomach or small intestine.

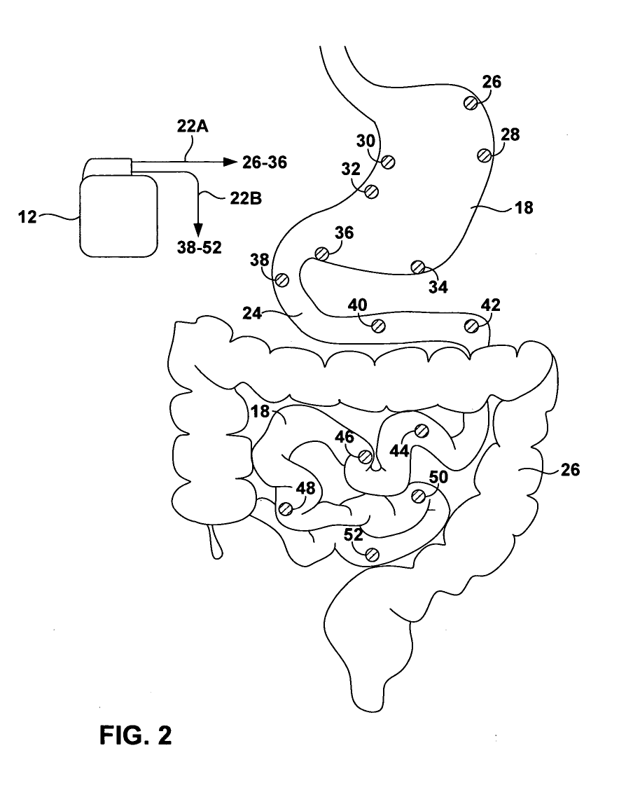

FIG. 2 is a schematic diagram illustrating a variety of stimulation sites on stomach 18 and small intestine 20, including duodenum 16. In the example of FIG. 2, stimulator 24 contains two leads 22A, 22B. Leads 22A, 22B may be tunneled through abdominal tissue in order to electrically couple stimulator 12 to desired stimulation sites. The distal ends of leads 22A, 22B are not shown in FIG. 2. Instead, each lead 22A, 22B is shown in conjunction with stimulation sites to which each lead is directed.

For example, lead 22A may be applied to any of stimulation sites 26, 28, 30, 32, 34 or 36 within stomach 18. Alternatively, two or more leads 22A may be applied to two or more stimulation sites 26, 28, 30, 32, 34 or 36 within stomach 18. Implantable stimulator 12 delivers a first set of stimulation pulses via lead 22A to induce a sensation of fullness or nausea, and thereby discourage food intake. Stimulation sites 26-36 vary from the proximal portion of the stomach 18, to the middle of the stomach, and to the exit of the stomach.

Lead 22B may be applied to any of stimulation sites 38, 40, 42, 44, 46, 48, 50 and 52 within small intestine 20. Alternatively, two or more leads 22A may be applied to two or more stimulation sites 38, 40, 42, 44, 46, 48, 50 and 52 within small intestine 20. Implantable stimulator 12 delivers a second set of stimulation pulses via lead 22B to increase motility in small intestine 20, and thereby reduce absorption of calories by patient 16 before food moves into the large intestine. Reduced absorption may be beneficial for patients who still eat too much food, despite induced fullness and nausea, but cannot afford to store the excess calories.

Any combination of stimulation sites shown in FIG. 2 may be utilized by a clinician depending on the patient's condition and desired treatment. In some embodiments, multiple leads may access multiple stimulation sites simultaneously. For example, two or more stimulation sites on each of stomach 18 and small intestine may be coupled to stimulator 12 via respective leads. Accordingly, in some embodiments, stimulator 12 may be coupled to four or more leads.

As an example, for a young patient who is otherwise healthy, a clinician may elect to implant two stomach leads to stimulator 12 to induce fullness or nausea, and implant two duodenum leads to increase motility of the small intestine 20. In contrast, for an obese patient who also has a condition that hinders normal motility within small intestine 18, the clinician may implant a larger number of leads for stimulation of the duodenum 24 or small intestine 20.

FIG. 2 shows a two-dimensional illustration depicting various stimulation sites on stomach 18 and small intestine 20. It should be noted that these locations may not only be on the ventral side of the organ. The stimulation sites where leads are attached indicate possible placements through the length of the organ. The actual placement may be on any side of the organ at that approximate position through the gastrointestinal tract. In addition, other locations on these organs may be appropriate to produce the desired effects from stimulation.

While the embodiment of FIG. 2 does not show any stimulation sites on large intestine 20, some embodiments may include stimulation of this organ in the gastrointestinal tract as well. In some cases, increased motility through the small intestine 18 may cause a backup of digested material upon reaching large intestine 20. Stimulation of the large intestine 26 may be included as part of the stimulation therapy in order for some patients to excrete digested material and avoid painful constipation. In particular, the stimulation also may increase motility of large intestine 26.

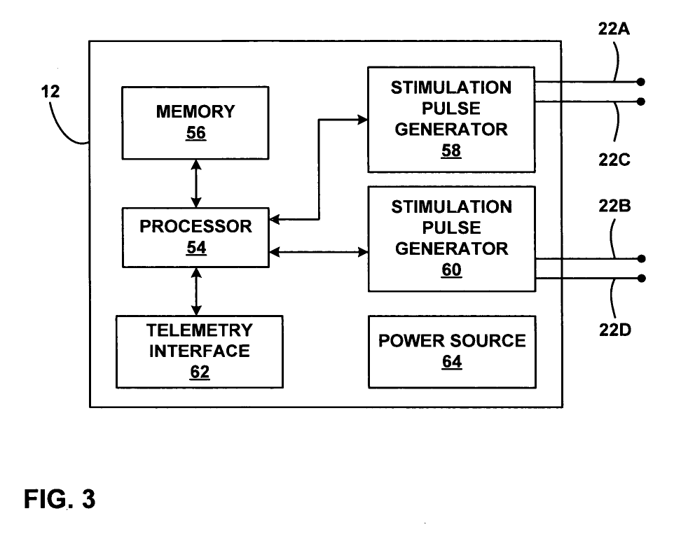

FIG. 3 is a functional block diagram illustrating various components of an implantable stimulator 12. Stimulator 12 includes a processor 54, memory 56, two or more stimulation pulse generators 58 and 60, wireless telemetry interface 62, and power source 64. Two stimulation pulse generators are utilized in the embodiment of FIG. 3 in order to provide different sets of stimulation pulses to the stomach 18 and small intestine 20.

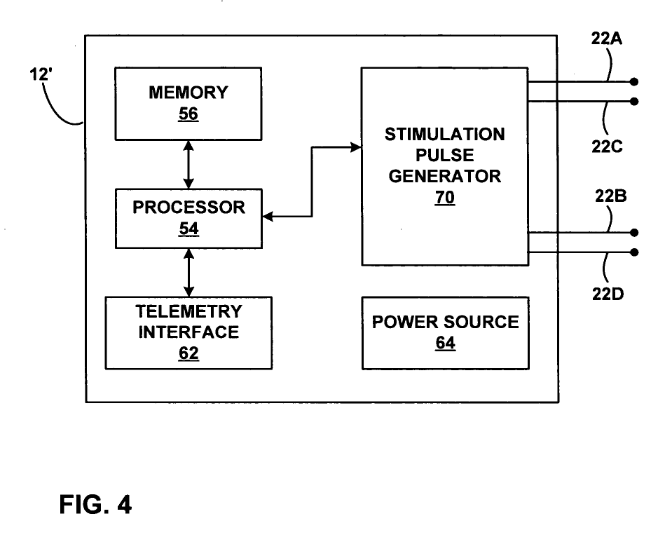

Electrical leads 22A, 22C extend from the stimulator housing and are connected to stimulation pulse generator 58 to apply stimulation pulses to stomach 14 to induce a sensation of fullness or nausea. Electrical leads 22B, 22D extend from the stimulator housing and are connected to stimulation pulse generator 60 to apply pulses to small intestine 18 to increase motility. Although pairs of leads 22A, 22C and 22B, 22D are shown in FIG. 4, a greater or lesser number of leads may be provided to deliver stimulation to different stimulation sites, or to achieve various bipolar, multipolar, and unipolar (“active can”) stimulation arrangements.

Memory 56 stores instructions for execution by processor 54 and stimulation therapy data. Stimulation information is recorded for long-term storage and retrieval by a user, or used in the adjustment of stimulation parameters, such as amplitude, pulse width or pulse rate. Memory 56 may include a single memory or separate memories for storing instructions, stimulation parameter sets, and stimulation information.

Processor 54 controls stimulation pulse generator 58 and stimulation pulse generator 60 in delivering first and second sets of electrical stimulation pulses. Processor 54 also controls telemetry interface 62 in exchanging information with external programmer 14. Based on stimulation parameters programmed by external programmer 14, processor 54 interprets the parameters to instruct appropriate stimulation by both stimulation pulse generators. The parameters which govern the pulses generated by each pulse generator 58, 60 may be different. In this example, stimulation pulse generator 58 is configured to generate stimulation pulses for delivery to stomach 14 while stimulation pulse generator 60 is configured to generate stimulation pulses to small intestine 18.

Stimulation pulse generator 58 provides electrical stimulation according to the stored parameter values for the stomach 18 via leads 22A, 22C carrying electrodes implanted at stimulation sites on stomach 18. Stimulation pulse generator 60 provides electrical stimulation according to the stored parameter values for the small intestine 20 via leads 22B, 22D carrying electrodes implanted on small intestine 18. The availability of two or more stimulation pulse generators 58, 60 facilitate separate control and delivery of first and second sets of stimulation pulses, and simultaneous delivery of the stimulation pulses, if desired. [00461 Wireless telemetry in stimulator 12 may be accomplished by radio frequency (RF) communication or proximal inductive interaction of implantable stimulator 12 with external programmer 14. Processor 54 controls telemetry interface 62 to exchange information with external programmer 14. Processor 54 may transmit operational information and sensed information to programmer 14 via telemetry interface 62. Also, in some embodiments, pulse generator 12 may communicate with other implanted devices, such as stimulators or sensors, via telemetry interface 62.

Power source 64 delivers operating power to the components of implantable stimulator 12. Power source 64 may include a battery and a power generation circuit to produce the operating power. In some embodiments, the battery may be rechargeable to allow extended operation Recharging may be accomplished through proximal inductive interaction between an external charger and an inductive charging coil within stimulator 12. In other embodiments, an external inductive power supply could transcutaneously power stimulator 12 whenever stimulation therapy is to occur.

FIG. 4 is a functional block diagram illustrating various components of another implantable stimulator 12′. Stimulator 12′ generally conforms to stimulator 12 of FIG. 3. For example, stimulator 12′ includes a processor 54, memory 56, telemetry interface 62, and power source 64. Instead of two or more stimulation pulse generators, however, stimulator 12′ incorporates a single pulse generator 70 that produces first and second sets of stimulation pulses on a time-interleaved basis for delivery to the stomach 18 and small intestine 20, respectively.

Stimulation pulse generator 70 may include a switching matrix, controlled by processor 54, that selectively couples the output of stimulation pulse generator 70 across leads 22A, 22C, or leads 22B, 22D to deliver either a first set of stimulation pulses to stomach 18 to induce a sensation of fullness or nausea, or a second set of stimulation pulses to small intestine 20 to increase motility. Again, although pairs of leads 22A, 22C and 22B, 22D are shown in FIG. 4, a greater or lesser number of leads may be provided to deliver stimulation to different stimulation sites, or to achieve various bipolar, multipolar, and unipolar (“active can”) stimulation arrangements.

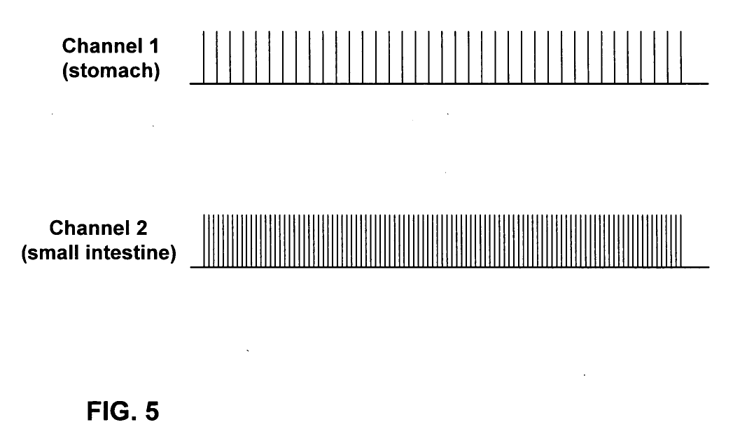

FIG. 5 is a timing diagram illustrating an example of continuous, asynchronous delivery of stimulation pulses on two separate “channels” to the stomach 18 and small intestine 20. On a first channel (Channel 1), stimulator 12 delivers a first set of stimulation pulses to stomach 18 via one or more leads 22 to induce a sensation of fullness or nausea. On a second channel (Channel 2), stimulator 12 delivers a second set of stimulation pulses to small intestine 20 via one or more leads 22 to increase motility. For each channel, stimulation is delivered as a continuous train of stimulation pulses, without any synchronization between channels, or any bursts of pulses. The channels are not synchronized to each other, as the pulses are delivered independently on Channels 1 and 2.

For the first set pulses delivered to stomach 18 (Channel 1), stimulation parameters are selected to induce a sensation of fullness or nausea and limit food intake. For example, the first set of stimulation pulses may have a pulse amplitude in a range of approximately 1 to 10 volts, a pulse width in a range of approximately 50 microseconds to 10 milliseconds, and a pulse rate in a range of approximately 1 to 100 Hz. For stimulation of stomach 18, the pulse rate is more preferably in a range of approximately 2 to 40 Hz, and even more preferably in a range of approximately 5 to 20 Hz. In the example of FIG. 5, the first set of pulses for stomach 18 is delivered at a rate of approximately 14 Hz to cause feelings of nausea. The terms pulse rate and pulse frequency may be used interchangeably in this description.

For the second set of pulses delivered to small intestine 20 (Channel 2), stimulation parameters are selected to increase motility within the small intestine and limit caloric absorption. Like the first set of stimulation pulses, the second set of stimulation pulses for small intestine 20 may have a pulse amplitude in a range of approximately 1 to 10 volts, a pulse width in a range of approximately 50 microseconds and to 10 milliseconds, and a pulse rate in a range of approximately 1 to 100 Hz. For stimulation of small intestine 20, the pulse rate is more preferably in a range of approximately 2 to 50 Hz, and even more preferably in a range of approximately 5 to 40 Hz. In the example of FIG. 5, the second set of pulses for the small intestine 20 is delivered at a pulse rate of approximately 40 Hz to increase motility in small intestine 20. In some embodiments, an instant start to delivery of the stimulation pulses may be provided. However, a gradual ramp up in stimulation intensity may be applied to prevent muscle shock and patient discomfort. This ramp may be in the form of a gradually increasing pulse rate, amplitude, or pulse width.

In the example of FIG. 5, the first and second sets of stimulation pulses delivered on Channels 1 and 2, respectively, are delivered continuously and independently of one another. Hence, there is no synchronization between pulses delivered to stomach 18 and pulses delivered to small intestine 20. In some embodiments, one or both of the sets of stimulation pulses may be temporarily turned OFF, either automatically or in response to a command entered by the patient 16 via programmer 14. For example, patient 16 may elect to turn off one or both of the sets of stimulation pulses at selected times, such as during sleep, or when the patient experiences significant discomfort. Likewise, in some embodiments, patient 16 may adjust the intensity of either set of the stimulation pulses. Also, stimulator 12 or programmer 14 may include a clock to selectively activate and deactivate stimulation at different times of the day.

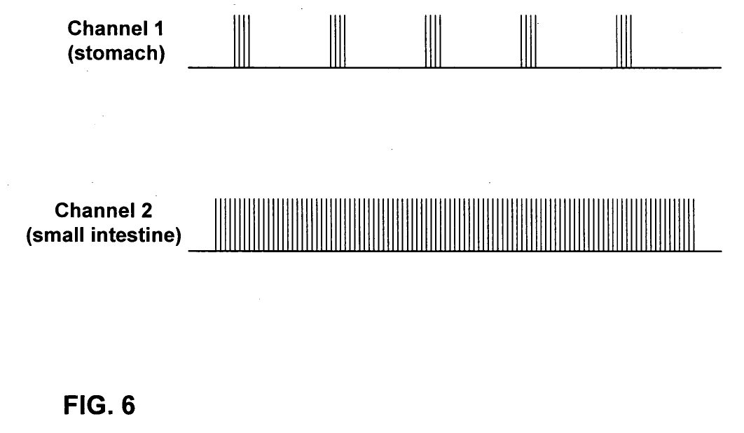

FIG. 6 is a timing diagram illustrating asynchronous delivery of stimulation on two separate channels to the stomach 18 and small intestine 20, with one channel delivering bursts of pulses. As shown in FIG. 6, the second set of stimulation pulses delivered on Channel 2 to small intestine 20 is delivered as a continuous train of pulses, as in the example of FIG. 5. The stimulation pulses delivered on Channel 2 in the example of FIG. 6 may have amplitudes, pulse widths and pulse rates similar to those identified above for the second set of pulses described with respect to FIG. 5. As further shown in FIG. 6, the first set of stimulation pulses delivered on Channel 1 to stomach 18 is delivered as a series of pulse bursts. In other embodiments, Channel 2 may deliver bursts of pulses to the small intestine 20 while Channel 1 delivers a continuous train of pulses to the stomach 18. Each burst is characterized by a pulse rate for pulses delivered within the burst, a burst rate, and a burst length.

The individual pulses in each burst may have amplitudes, pulse widths, and pulse rates similar to those identified above for the first set of pulses described with respect to FIG. 6. For example, each burst may carry a set of pulses delivered at a rate of 1 to 100 Hz. In the example, of FIG. 6, each burst contains pulses delivered at a rate of approximately 40 Hz. The burst rate may be in a range of approximately 3 to 15 bursts per minute, which is approximately 1 to 5 times the typical gastric slow wave frequency in a healthy patient. The burst length may be in a range of approximately 10 to 50 percent of the burst period, i.e., the period between successive bursts.

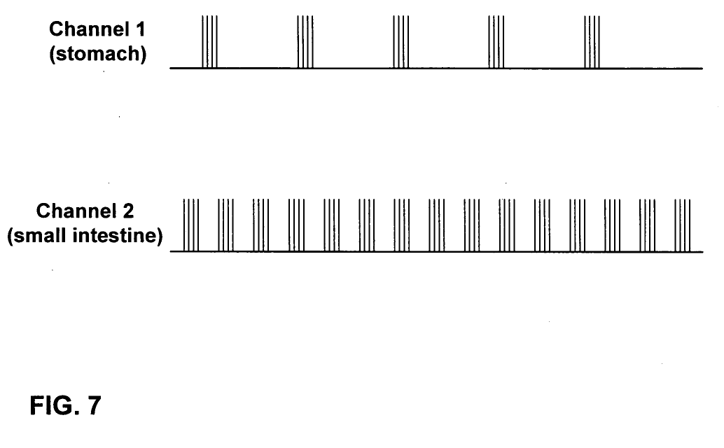

FIG. 7 is a timing diagram illustrating asynchronous delivery of stimulation on two separate channels to the stomach and small intestine, with both channels delivering bursts of pulses. As shown in FIG. 7, bursts of pulses are delivered on both Channels 1 and 2. However, in the example of FIG. 7, the bursts on Channels 1 and 2 are not synchronized with one another. In general, the pulses within the bursts delivered on Channels 1 and 2 may have parameters similar to those described with respect to FIG. 5. For example, the pulses may have a pulse amplitude in a range of approximately 1 to 10 volts, a pulse width in a range of approximately 50 microseconds to 10 milliseconds, and a pulse rate in a range of approximately 1 to 100 Hz.

In some embodiments, the pulse parameters may be different for the different Channels. The pulse rate for Channel 1 may be lower than the pulse rate for Channel 2. As in the particular example of FIG. 5, the first set of pulses for stomach 18 may be delivered on Channel 1 at a rate of approximately 14 Hz to cause feelings of fullness or nausea, while the second set of pulses for the small intestine 20 may be delivered at a pulse rate of approximately 40 Hz to increase motility in small intestine 20.

In addition, the burst parameters associated with Channels 1 and 2 may be different. For example, as in FIG. 7, the bursts delivered to stomach 18 on Channel 1 may have a burst rate of approximately 3 to 15 bursts per minute, and a burst length in a range of approximately 10 to 50 percent of the burst period. The bursts delivered to small intestine 20 on Channel 2 may have a burst rate in a range of approximately 8 to 50 bursts per minute, and a burst length in a range of approximately 10 to 50 percent of the burst period.

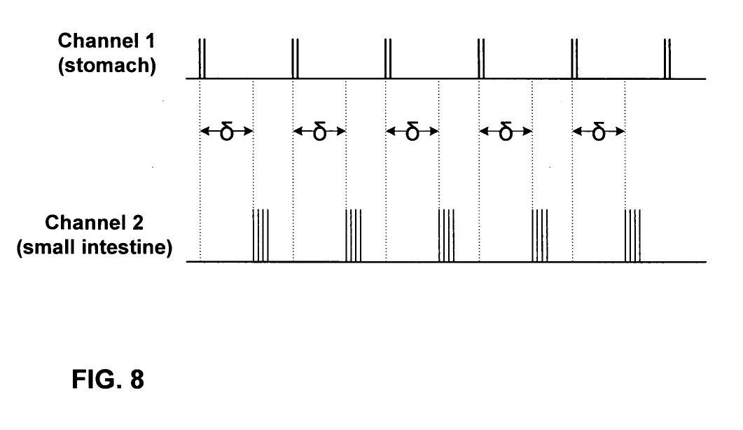

FIG. 8 is a timing diagram illustrating synchronous delivery of stimulation on two separate channels (Channels 1 and 2) to the stomach 18 and small intestine 20, with both channels delivering bursts of pulses. In the example of FIG. 8, the bursts delivered to stomach 18 on Channel 1 and to small intestine 20 on Channel 2 are synchronized with one another. In particular, there is a time delay 6 between the delivery of each burst on Channel 1, and the delivery of a corresponding burst on Channel 2. The bursts delivered on Channel I and Channel 2 are synchronized to each other to link the smooth muscle contractions in both the stomach 18 and small intestine 20. In some patients, a synchronized stimulation technique may be more effective in achieving weight loss.

The pulses delivered in the Channel 1 and Channel 2 bursts may have amplitude, pulse width, and pulse rate parameters similar to those described above. For example, the pulses may have a pulse amplitude in a range of approximately 1 to 10 volts, a pulse width in a range of approximately 50 microseconds to 10 milliseconds, and a pulse rate in a range of approximately 1 to 100 Hz. However, the pulse parameters may be different for channel 1 and channel 2. In addition, the Channel 1 and Channel 2 stimulation may have similar burst parameters. For example, the bursts delivered to stomach 18 on Channel 1 may have a burst rate of approximately 3 to 15 bursts per minute, and a burst length in a range of approximately 10 to 50 percent of the burst period. In light of the synchronization of Channel 1 and Channel 2, the bursts delivered to small intestine 20 on Channel 2 may have a burst rate that is identical or substantially similar to the burst rate on Channel 1. However, the Channel 1 and 2 bursts may have different burst lengths.

For synchronous stimulation, stimulator 12 triggers the delay of Channel 2 bursts in synchronization with Channel 1 bursts, or vice versa. In the example of FIG. 8, each burst on Channel 2 is delivered to small intestine 20 at a time delay δ following delivery of a burst to stomach 18 on Channel 1. Hence, there may be a one-to-one correspondence between Channel 1 and Channel 2 bursts, albeit on a time-delayed basis. In other embodiments, however, each Channel 1 burst may trigger two or more Channel 2 bursts on a time-delayed basis. The time delay 6 may be any period of time that separates the beginning of a burst on Channel 1 to a burst on Channel 2. The time delay δ will be shorter than the longest burst frequency. However, the time delay δ may be a few milliseconds or a relatively long period of time on the order of several seconds.

In some embodiments, bursts of stimulation pulses delivered to the stomach 18 and small intestine 20 may be synchronized with sensed gastric slow waves within the stomach and small intestine, respectively. For example, delivery of stimulation pulses to stomach 18 may be triggered when the gastric slow wave in the stomach crosses a predetermined threshold. Similarly, delivery of stimulation pulses to small intestine 20 may be triggered when the gastric slow wave in the small intestine crosses a predetermined threshold. The normal slow wave in stomach 18 is ordinarily on the order of 3 cycles per minute, while the normal slow wave in small intestine 20 is on the order of ten cycles per minute.

Various embodiments of the described invention may include processors that are realized by microprocessors, Application-Specific Integrated Circuits (ASIC), Field-Programmable Gate Arrays (FPGA), or other equivalent integrated or discrete logic circuitry. The processor may also utilize several different types of data storage media to store computer-readable instructions for device operation. These memory and storage media types may include any form of computer-readable media such as magnetic or optical tape or disks, solid state volatile or non-volatile memory, including random access memory (RAM), read only memory (ROM), electronically programmable memory (EPROM or EEPROM), or flash memory.

Many embodiments of the invention have been described. Various modifications may be made without departing from the scope of the claims. These and other embodiments are within the scope of the following claims.

1. A method for electrical stimulation of a gastrointestinal tract of a patient, the method comprising:
generating a first set of electrical stimulation pulses;
generating a second set of electrical stimulation pulses;
applying the first set of pulses to a stomach of the patient to limit food intake; and
applying the second set of pulses to a small intestine of the patient to increase motility.

2. The method of claim 1, further comprising applying the first and second sets of electrical pulses substantially continuously.

3. The method of claim 1, further comprising generating the first set of pulses at a pulse rate of approximately 1 to 100 Hz.

4. The method of claim 1, further comprising generating the first set of pulses at a pulse rate of approximately 5 to 20 Hz.

5. The method of claim 1, further comprising applying the first set of pulses in bursts at a burst rate of approximately 3 to 15 bursts per minute.

6. The method of claim 5, wherein each of the bursts has a burst length of approximately ten to fifty percent of a period between successive bursts.

7. The method of claim 1, further comprising generating the second set of pulses at a pulse rate of approximately 5 to 40 Hz.

8. The method of claim 1, further comprising applying the second set of pulses in bursts at a burst rate of approximately 8 to 50 bursts per minute.

9. The method of claim 8, wherein each of the bursts has a burst length of approximately ten to fifty percent of a period between successive bursts.

10. The method of claim 1, further comprising applying the first set of pulses in bursts at a burst rate of approximately 3 to 15 bursts per minute, and applying the second set of pulses in bursts at a burst rate of approximately 8 to 50 bursts per minute.

11. The method of claim 1, further comprising applying the first set of pulses in bursts, and applying the second set of pulses substantially continuously.

12. The method of claim 1, further comprising applying the first set of pulses in first bursts, and applying the second set of pulses in second bursts, wherein each of the second bursts follows one of the first bursts by a predetermined time delay.

13. The method of claim 1, further comprising applying the first set of pulses via one or more implanted electrodes coupled to the stomach, and applying the second set of pulses via one or more implanted electrodes coupled to the small intestine.

14. The method of claim 1, wherein the first set of pulses have pulse parameters selected to induce a sensation of fullness of nausea and thereby limit food intake.

15. The method of claim 1, wherein the second set of pulses have pulse parameters selected to accelerate movement of food through the small intestine.

16. A device for electrical stimulation of a gastrointestinal tract of a patient, the device comprising:
a first lead carrying a first electrode;
a second lead carrying a second electrode; and
a pulse generator that delivers a first set of electrical stimulation pulses via the first lead and a second set of electrical stimulation pulses via the second lead,
wherein the first set of pulses are formulated for delivery to a stomach of the patient to limit food intake, and
wherein the second set of pulses are formulated for delivery to a small intestine of the patient to increase motility.

17. The device of claim 16, wherein the pulse generator delivers the first and second sets of electrical pulses substantially continuously.

18. The device of claim 16, wherein the pulse generator delivers the first set of pulses at a pulse rate of approximately 1 to 100 Hz.

19. The device of claim 16, wherein the pulse generator delivers the first set of pulses at a pulse rate of approximately 5 to 20 Hz.

20. The device of claim 16, wherein the pulse generator delivers the first set of pulses in bursts at a burst rate of approximately 3 to 15 bursts per minute.

21. The device of claim 20, wherein each of the bursts has a burst length of approximately ten to fifty percent of a period between successive bursts.

22. The device of claim 16 wherein the pulse generator delivers the second set of pulses at a pulse rate of approximately 5 to 40 Hz.

23. The device of claim 16, wherein the pulse generator delivers the second set of pulses in bursts at a burst rate of approximately 8 to 50 bursts per minute.

24. The device of claim 23, wherein each of the bursts has a burst length of approximately ten to fifty percent of a period between successive bursts.

25. The device of claim 16, wherein the pulse generator delivers the first set of pulses in bursts at a burst rate of approximately 3 to 15 bursts per minute, and delivers the second set of pulses in bursts at a burst rate of approximately 8 to 50 bursts per minute.

26. The device of claim 16, wherein the pulse generator delivers the first set of pulses in bursts, and delivers the second set of pulses substantially continuously.

27. The device of claim 16, wherein the pulse generator delivers the first set of pulses in first bursts, and delivers the second set of pulses in second bursts, wherein each of the second bursts follows one of the first bursts by a predetermined time delay.

28. The device of claim 16, wherein the pulse generator includes a first pulse generator to deliver the first set of pulses and a second pulse generator to deliver the second set of pulses.

29. The device of claim 16, wherein the first set of pulses have pulse parameters selected to induce a sensation of fullness of nausea and thereby limit food intake.

30. The device of claim 16, wherein the second set of pulses have pulse parameters selected to accelerate movement of food through the small intestine.

31. A device for electrical stimulation of a gastrointestinal tract of a patient, the device comprising:
means for generating a first set of electrical stimulation pulses;
means for generating a second set of electrical stimulation pulses;
means for applying the first set of pulses to a stomach of the patient to limit food intake; and
means for applying the second set of pulses to a small intestine of the patient to increase motility.
